(19) United States
(12) Patent Application Publication
(10) Pub. No.: US 2024/0024601 A1
(43) Pub. Date: Jan. 25, 2024

(54) DEVICE AND METHOD FOR BREATHING ASSISTANCE

(71) Applicant: QUADRUS MEDICAL TECHNOLOGIES, INC., White Plains, NY (US)

(72) Inventors: Nicolas Wadih Chbat, Ardsley, NY (US); Caitlyn Marie Chiofolo, New Hyde Park, NY (US)

(21) Appl. No.: 18/256,745

(22) PCT Filed: Dec. 9, 2021
(86) PCT No.: PCT/US2021/062711

§ 371 Date: Jun. 9, 2023

Related U.S. Application Data
(60) Provisional application No. 63/123,191, filed on Dec. 9, 2020.

(51) Int. Cl. A61M 16/00; G16H 50/50; G16H 50/70; A61M 16/10; A61M 16/16

(52) U.S. Cl. CPC A61M 16/024; G16H 50/50; G16H 50/70; A61M 16/1055; A61M 16/1075; A61M 16/16; A61M 16/0003; A61M 2016/0027; A61M 2016/1025; A61M 2205/18; A61M 2205/505; A61M 2205/84

(57) ABSTRACT

A breathing assistance system includes a control unit configured to be connected to a device, the device configured to provide breathing assistance to a patient, the control unit configured to simultaneously control parameters that include a pressure of air supplied to the patient, and at least one of a volume and a flow rate of the air.

### CROSS REFERENCE TO RELATED APPLICATIONS

This application claims the benefit of U.S. Provisional Application No. 63/123,191, filed Dec. 9, 2020 which is incorporated herein by reference in its entirety.

### BACKGROUND

The present invention generally relates to breathing assistance for medical patients. More specifically, the present invention relates to ventilator devices and methods that provide for both pressure and volume control, as well as real time patient monitoring and adjustment of ventilator variables.

Ventilators are life-supporting devices, which help patients breathe when they cannot on their own. Without these mechanical ventilators, the patients would certainly die, but with them sometimes they cause ventilator-induced lung injury (VILI), such as volutrauma or barotrauma. Around 24% of ventilated patients suffer from VILI globally. Besides its function of delivering O₂ to the lungs, the ventilator should also aid in liberating the patient from it (the machine), i.e., stopping the patient's dependency on the ventilator to respirate, and instead help the patient build eventual energy to do the work of breathing on his/her own.

### SUMMARY

An embodiment of a breathing assistance system includes a control unit configured to be connected to a device, the device configured to provide breathing assistance to a patient, the control unit configured to simultaneously control parameters that include a pressure of air supplied to the patient, and at least one of a volume and a flow rate of the air.

An embodiment of a breathing assistance method includes receiving sensor data related to a breathing assistance device at a control unit, the device configured to provide breathing assistance to a patient, and simultaneously controlling parameters that include a pressure of air supplied to the patient, and at least one of a volume and a flow rate of the air.

An embodiment of a computer program product includes a storage medium storing instructions executable by a processor to perform a method of facilitating use of a breathing assistance device by a patient. The method includes receiving patient data from measurements of a patient, and device data related to operation of the breathing assistance device, displaying information to the patient, the information including patient characteristics, and device settings including values of the parameters, and displaying one or more recommendations to a user based at least one of the patient characteristics.

An embodiment of a testing system for testing a breathing assistance device includes a testing assembly configured to emulate patient breathing activity, the testing assembly configured to be connected to the breathing assistance device, one or more sensors configured to measure one or more properties of air flow produced by the testing assembly, and a control unit in communication with the testing assembly, the control unit configured to control operation of the testing assembly.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is a block diagram of an embodiment of a ventilator;

FIG. 2 is a block diagram illustrating functional aspects of an embodiment of a ventilator, including a control strategy implemented by one or more controllers;

FIG. 3 depicts an embodiment of a ventilator configured to operate according to a mathematical lung model;

FIG. 4 depicts an embodiment of a ventilator configured to operate according to a model and an optimization routine;

FIG. 5 depicts an embodiment of a ventilator configured to control ventilator variables based on demographic variables of a patient;

FIG. 6 is a hydraulic diagram representing hydraulic functionality of an embodiment of a ventilator;

FIG. 7 is a block diagram of a control coupling of an embodiment of a ventilator;

FIG. 8 depicts an embodiment of a ventilator configured to ventilate two patients simultaneously.

FIG. 9 depicts an example of information presented by a display included in or connected to an embodiment of a ventilator;

FIG. 10 depicts another example of information presented by the display of FIG. 9;

FIG. 11 depicts a further example of information presented by the display of FIGS. 9 and 10;

FIG. 12 depicts an example of settings limits and adjustments information presented by a display of an embodiment of a ventilator;

FIG. 13 depicts an example of physiologic, temporal, breath-based, therapeutic goal-driven, calculated or estimated information presented by a display included in or connected to an embodiment of a ventilator;

FIG. 14 depicts another example of information presented by the display of FIG. 13;

FIG. 15 depicts a further example of information presented by the display of FIGS. 13 and 14;

FIG. 16 depicts an example of trending information presented by a display included in or connected to an embodiment of a ventilator; and

FIG. 17 depicts an embodiment of a testing system for evaluating a ventilator and/or other breathing assistance device.

### DETAILED DESCRIPTION

Devices, systems and methods for breathing assistance are described herein. Embodiments include a ventilator configured to provide breathing assistance for a patient (e.g., breathing for the patient or assisting a patient's breathing), which includes functionality for controlling both air (and O₂ mixture) pressure and volume during operation. This pressure and volume control may be referred to as pressure controlled flow actuated (PCFA) ventilation. It resembles another mode called PRVC (pressure regulated volume control) but differs from it in ways that are described below. The ventilator may be configured to provide breathing assistance to a single patient, or provide breathing assistance to multiple patients (using the same ventilator).

Ventilators described herein provide a number of advantages in comparison to conventional ventilators. Existing breathing assistance devices typically provide one of two modes of breathing assistance: pressure-controlled ventilation (PCV), which is typically used to breathe for a patient, and flow-controlled ventilation (FCV), which is typically used to assist a patient's breathing. An example of PCV is the aforementioned PRVC, which checks tidal volume and actuates air pressure based on the tidal volume. An example of FCV is Average Volume Assured Pressure Support (AVAPS), which is used for non-invasive ventilation (NIV), and is typically used in BiPAP machines.

The ventilators described herein provide for both pressure and volume control, which has the benefits of PCV while additionally providing flow and/or volume control. This allows not only a single machine to be used for both types of breathing assistance control, but also a single mode of ventilation, which is used to control both flow to and pressure at the patient (connection).

The ventilators described herein allow for single-patient and multi-patient ventilation (on the same ventilator). Such ventilators feature control loops that can be customized for each ventilated patient, which avoids excessive tidal volume. With the use of the single PCFA ventilator mode, these multi-patient capable machines include at least functionality of PCV, while additionally controlling (and adequately distributing) flow to each patient with the use of valves. These multi-patient capable machines include at least functionality for PCV, while having a smaller form as compared to conventional machines. The smaller form provides various benefits, such as ease of transport, increased ability for use in emergency situations, minimal presence at bedside, and ease of stockpile/storage. In addition, embodiments described herein offer simpler design (e.g., simpler and more intuitive user interfaces), and simpler operation. These benefits make it easier for medical practitioners to use, and may also allow for use by lay persons.

The ventilators described herein may be configured as “smart” ventilators, which operate according to a closed-loop control regime. Both pressure and volume (flow rate) can be adjusted in real time based on setpoints selected for a specific patient or patient demographic. In addition, the control regime may utilize equations, models or other patient simulations to provide real time setpoints and permit control based on individual patients. For example, ventilator settings/variables can be monitored and adjusted in real time by a controller or other processor, which reduces the number of modes and reduces the need for physicians and other practitioners to actively control machine variables.

Another advantageous feature of the ventilators described herein, is that the ventilators can be modular, having a base configuration and modules that can be added based on situational and patient needs. In this way, base models can be easily configured for different uses (e.g., during transport, in hospital, at home). The ventilator model, controller, and estimation algorithms can be applied in multiple embodiments, such as a transport ventilator (for which modules can be provided that provide or have stability to support use during transport) and a home ventilator (where modules such as an oxygen concentrator or blender can be provided).

Embodiments of the ventilator and/or breathing assistance device described herein are able to be operated by medically trained personnel (e.g., in hospitals or healthcare facilities) and/or by lay operators (anyone) in home use, ambulatory/transport/emergency settings, or any other setting. The embodiments can have various levels of sophistication (e.g., different levels of automatic control).

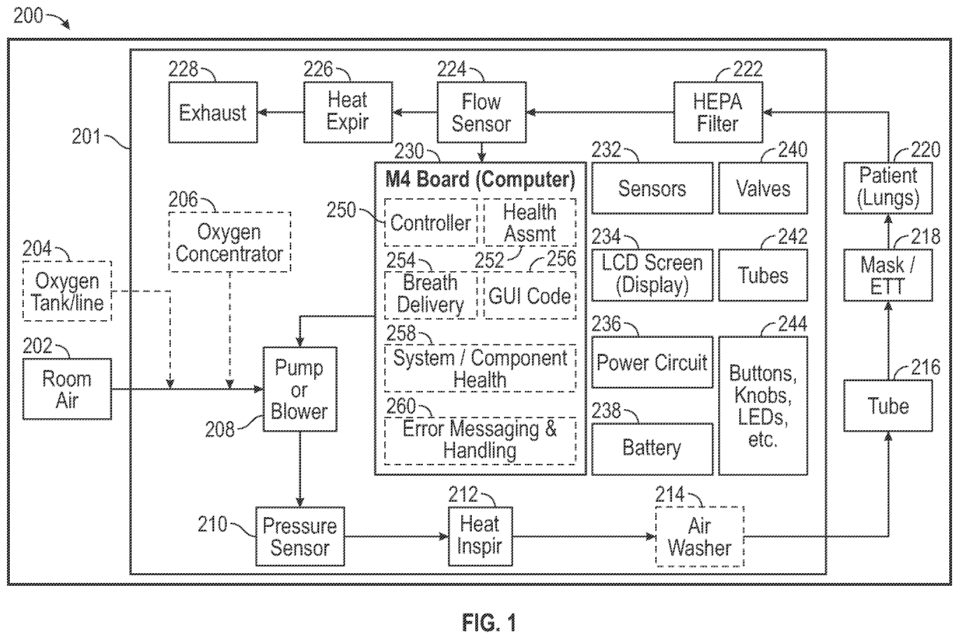

FIG. 1 depicts an embodiment of a ventilator 200. The ventilator 200 can come in different categories, such as invasive or non-invasive air delivery. Other categories may include various levels of intelligence. For example, operational variables (e.g., oxygen concentration, volume, flow rate, pressure, etc.) can be manually controlled, automatically controlled, or controlled in conjunction with a human operator (e.g., by providing suggestions or requesting approval of variable changes). For example, the ventilator 200 may be a “smart” ventilator that includes software that enables personalized ventilation, e.g., via patient-disease models. Other categories include local operation, or remote operation. Remote operation can be accomplished in any suitable manner, such as via APIs accessible via WLAN, wifi, Bluetooth, and the like. Physical features may allow for remote operation, such as a detachable touchpad or screen, a display port for connection of an external monitor, or a remote control panel communicating with the ventilator 200 over radio frequency or equivalent medium.

For example, the ventilator is an invasive ventilator (e.g., operated by a trained medical professional in a hospital via endotracheal tube (intubation) air delivery), or a noninvasive smart ventilator (e.g., operated by anyone via mask (or helmet) air delivery).

FIG. 1 is a functional block diagram of an embodiment of the ventilator 200, which includes various features and components, one or more of which can be optional depending on the ventilator version and category.

The ventilator 200 includes a housing 201 that houses various components. As shown, some components are internal to the housing 201, and some components are external.

The ventilator 200 is connected to an air supply, such as room air 202, and connected to an oxygen supply via a tank/line 204. Air and oxygen enter the ventilator 200, are mixed with a valve (depending on the desired or set fraction of inspired oxygen FiO₂) and the mixture (referred to collectively as “air”) continues to a pump or blower 208. A pressure sensor 210 measures the pressure of the air from the blower 208. As discussed further below, any number of sensors 232 can be used to measure a number of variables (e.g., oxygen concentration, temperature, pressure, flow rate, etc.) The ventilator 200 can house an internal heater 212 and air washer 214 to condition the air, or an external heat/humidification device can be used. The air goes to/from a patient 220 via tubing 216, and a mask or endotracheal tube 218 (depending on whether an invasive or noninvasive mode is used). The patient's expired air returns to the ventilator 200 and is put through a HEPA filter 222 to remove harmful particles, moisture, or larger microorganisms, before being optionally heated by a heater 226 to kill harmful smaller microorganisms and viruses (e.g., if the HEPA filter 222 does not sufficiently kill such microorganism and viruses) and exhausted 228. Flow rate may be measured via a flow sensor 224, and any additional variables may be measured via appropriate sensors 232 (e.g., internal to the housing 201 or external, e.g., as modular components).

It is noted that the ventilator 200 is not limited to the above embodiment. The ventilator 200 may include all or some of the above components in various configurations. Components may be internal to the housing and provide a base version of the ventilator 200, and various additional components may be provided as modular components to customize the ventilator for various purposes and desired capabilities.

For example, medical air and oxygen from the wall (in some institutions) or in tanks can serve as the sources of pressurized air. In addition, optional modules can include physical modules such as heating systems, humidification systems, blowers (e.g., pump or blower 208), compressors, and internal oxygen concentrators (e.g., an oxygen concentrator 206). For example, a blower may not be needed in a critical care ventilator connected to one or more pressurized gas lines, but a blower may be required for a home ventilator connected to an ambient air supply, with or without the use of a compressor. Other modules include software modules such as modules for concurrent ventilation of multiple (e.g., two) patients, different breath trends (including but not limited to alveolar compliance, tidal volume, work of breathing and pressure momentum (pressure time product)), different personalized ventilation settings (based on, e.g., physiological models), and/or different noninvasive disease assessments. Ventilators such as the ventilator 200 can come with or without these optional modules, depending on their intended use (e.g., hospital need, operation environment, target patient population, operator).

Additional components may be included, in the housing 201 or externally. For example, the ventilator 200 includes a display 234, a power circuit 236 connected to a battery 238 (or an external power supply), valves 240, tubes 242, interface devices 244 (e.g., buttons, control knobs, a touchscreen, etc.), and safety and alarming components.

Versions or categories of the ventilator are described as follows as embodiments in conjunction with flow diagrams. In the following, intubation and external compressed (medical) air and O₂ supply line sources are assumed, however the embodiments are not so limited.

As is demonstrated in the various embodiments, the ventilator 200 can be designed and implemented such that different modules can be separated from each other. In one embodiment, the ventilator 200 has a base version having basic characteristics that are standards across all versions and categories (like the PCFA) and other optional modules. In one embodiment, the base version and/or other versions (including modular components) are configured to operate according to a control strategy or method as described below.

The ventilator 200 includes or is connected to the one or more controllers, which may be in the form or any suitable processing device or processor. As described herein, a “controller” refers to a processing device that executes a closed-loop control regime that relies on a sensory feedback in order to command actions to an actuator, which could be a valve. The control regime may be used to adjust ventilator settings, which are also referred to as “ventilator variables,” examples of which include pressure, volume, flow rate, oxygen concentration, temperature, humidity, etc. The control regime may be based on various “patient variables,” which are physical properties, or parameters, of individual patients, and/or of classes of patients (e.g., demographics). Examples of patient variables include demographic information (e.g., height, weight, gender, etc.), disease information, physiological measurements (e.g., inspired/expired air flow or pressure, temperature, blood oxygen, blood carbon dioxide, blood pH, etc.) and others. Examples of patient parameters include airway resistance, lung compliance, chest wall compliance, arterial stenosis, epithelial barrier resistance, etc.

For example, the ventilator 200 houses a processing device 230 that includes one or more controllers 250 for performing various control functions. The processing device 230 receives ventilator variable data, such as flow measurements from the flow sensor 224, air temperature measurements, pressure measurements from the pressure sensor 210, and others. These measurements are used to control variables such as a flow rate and pressure. The processing device may also receive patient data, such as demographic data and physiological data from the patient, which may also be used to control variables.

In this example, the processing device 230 is configured to perform various functions, which may be performed by the controller 250 and/or individual control modules. The functions include health assessment 252, breath delivery control 254, user interface control 256, ventilator system and component monitoring 258, and error messaging and handling 260.

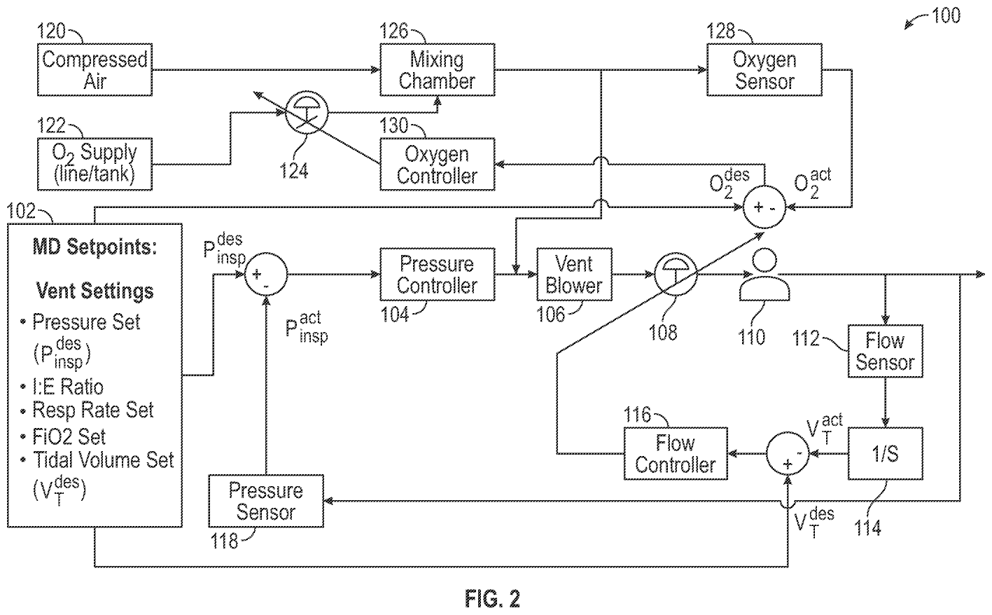

FIG. 2 illustrates aspects of a control strategy 100 implemented by one or more controllers (which may be embodied as one or more computer program products). In this example, the ventilator 200 includes or is connected to controllers that include a pressure controller (PC) 104, flow controller (FC) 116, and/or possibly an oxygen mixing controller (OC) 130. A patient 110 (the patient's lungs and tubing between the actuator and the patient, or plant of the control system) is connected to the ventilator, and closed-loop control is implemented in which operational variables and the patient are monitored and provided as feedback for variable control.

The pressure controller 104 provides feedback pressure control to avoid over- (or under-) pressurizing the lungs. The pressures in an inspiratory line will hence be under control to avoid damage (e.g., barotrauma) and fine-tune the air supplied to the patient 110. For unforeseen reasons, and in case of malfunction, a pressure release valve may be included.

The oxygen controller 130 provides feedback oxygen mixing control to avoid over- or under-oxygenation of the lungs. It ensures the regulation of the selected amount of inspired fraction of O₂ to be administered to the patient.

The flow controller 116 provides feedback flow control to avoid over-(or under-) distention of the alveoli. As such, damage such as volutrauma can be avoided. The flow controller 116, along with personalization of the pressure trajectory in real-time, ensures the regulation of the tidal volume (V_{T}).

In one embodiment, the control strategy utilizes various setpoints, which specify desired variables. The setpoints may be selected based on patient variables such as patient demographics, type and severity of a disease or medical condition, and/or physiological measurements of a specific patient. The setpoints may be selected values or ranges of values, or may be selected rates of change (trajectory) of values.

In use, for example, an operator (e.g., clinician) establishes setpoints or trajectories from user inputs selected via a human-computer interface 102 (e.g., user interface, knobs, buttons, etc.). In the example, the user inputs are directly used to establish target (targ) or desired (des) values (setpoints) or trajectories (values over time) for pressure, flow, and oxygen. Other embodiments are envisioned to have model-based determination of target or desired values/trajectories. Other embodiments are envisioned to adjust target or desired values/trajectories based on patient's desire to breathe spontaneously and/or upon detection of certain conditions (e.g., patient-ventilator dyssynchrony, early/late breath termination, double triggering, breath stacking, etc.); or based on the user's experience, or based on an AI algorithm that learns from previous patients (demographic classes, diagnoses, ventilator settings, and subsequent outcomes).

The desired variables (e.g., pressure desired Pᵢₙₛₚ^{des}, volume desired V_{T}^{des} and/or oxygen concentration desired O₂^{des}) are compared with actual (act) values/trajectories that are measured or observed via sensors. The difference between a desired or target value and a measured value is calculated as an error value (error), which is used as input to a respective controller or controllers. The controller(s) transforms the error to an input stimulus for an actuator. The controller is designed such that to abide by some requirements or specifications. For instance, the pressure curve should loyally follow the desired pressure trajectory as set by the physician with a maximum of 1% overshoot and in less than 100 msec.

For example, setpoints and measurements are used by the flow controller 116, the pressure controller 104, and/or the optional oxygen controller 130 to calculate errors (or errors are fed to controllers). The flow controller 116 receives an error signal or error value based on a comparison between flow rate measured by a flow sensor 112 and a setpoint. The sensed flow signal is sent to an integrator 114 to obtain the volume, and adjusts (as needed) a blower 106 or a valve such as a butterfly valve or proportional solenoid (PSol) 108 to eliminate or reduce the error. Alternatively, one or more valves connected to a pressurized air supply can be adjusted. The oxygen controller 130 in turn adjusts an O₂ supply 122 via, e.g., a valve or solenoid 124., based on an error between a setpoint and measurements from the oxygen sensor 128. In case of multiple gas tanks then the mixture of the gases needs to abide by the O₂^{des} command.

The pressure controller 104 receives an error signal or error value based on a comparison between pressure measured by a pressure sensor 118 and a setpoint, adjusts a blower 106 or a valve such as a butterfly valve or proportional solenoid (PSol) 108 accordingly.

The setpoint of the pressure could be a trajectory in time (e.g., square wave, or a double exponential (one for inspiration, one for expiration, if any)) that is continually adjusted according to an adaptive control law, whereby it adapts in real time based on deviation of V T from its target value and based on changing patient's lung characteristics.

The patient lung's characteristics could simply be the airway resistance and the lung compliance. These can be estimated in real time based on a dynamic equation of motion and by utilizing measurements of pressure and flow, and/or via other models such as a physics based model such as a dynamic equation of motion, or via a data-based model.

In this example, the ventilator's air supply is compressed air 120 and an oxygen (O 2) supply 122 tank or line. Oxygen travels through a valve 124 to a mixing chamber 126, or is dynamically mixed (without needing a mixing chamber) as air is supplied to a patient. Oxygen concentration of the air leaving the chamber 126 is sensed by an oxygen sensor 128 and compared to the FiO₂ setting (or oxygen concentration desired), the error of which is input to the oxygen controller 130, which controls the oxygen concentration via actuation of the valve 124 on the O₂ line. In other embodiments, the ventilator 200 can have a blower to suck ambient air into an oxygen concentrator (not shown), or a series of sieves to reduce the nitrogen in the air, before entering the chamber 126. This chamber may in some embodiments be an air compressor. In other embodiments, there could additionally be a pressure-release valve (not shown) which reduces high pressures above a given setpoint, and the pressure could be sensed additionally or alternatively by a pressure sensor placed after the mixing chamber 126, oxygen concentrator, oxygen compressor, or other chamber to actuate valves on one or both of the compressed air 120 and an oxygen (O₂) supply 122 lines. In other embodiments, oxygen concentration may be adjusted based on a desired concentration using one or more flow controllers and/or a mixing equation. Other embodiments may additionally include a temperature sensor or carbon dioxide sensor, or separate wall supplies or tanks of oxygen and medical air and oxygen from a wall line.

Each controller accomplishes their respective goals by controlling an actuator. For example, the pressure controller 104 actuates blower 106 speed (or a PSol in case wall gas lines are used), while the flow controller 116 and the oxygen controller 130 each actuates a valve 108, 124 (butterfly valve or a PSol). In another example, the flow controller 116 actuated one or more inspiratory valves, and the pressure controller 104 actuates one or more expiratory valves (the inspiratory and/or expiratory valve may be proportional type valves). In a modern ICU application, the pressure controller can actuate an expiratory valve. All closed-loop controllers in this example are error driven, meaning that an error signal (difference between a desired setpoint/trajectory and actual sensor value) is transformed into a current (or a voltage) that drives the corresponding actuator. This transformation is accomplished according to a control law specifically designed to transform the error into an actuator input.

The controllers may be configured according to various control laws or algorithms. For example, a controller may be a linear (1^{st} or higher order pole) controller, or in its simplest form, belong to a proportional-integral (PI) control law family, where the controller's gains (controller parameters) are carefully designed based on the corresponding plant (or dynamic system—here, lungs and tubing) that it is controlling and on the sources of disturbances that it may encounter. The control laws include rules for following set points/trajectories and/or rules for rejecting disturbances. The stability of each of the three controllers is also proven. Robustness is shown by varying the range of parameters of the plants while still adhering to stability criteria like the Nyquist and the Circle criteria.

In some embodiments, a pressure controller actuates a valve (e.g., proportional solenoid or valve) in the expiratory line based on a sensor in the expiratory (or inspiratory) line, while inspiratory line flow controllers actuate other valves to ensure desired oxygen concentration and flow that does not cause excess tidal volume.

Limiting the V_{T} (tidal volume) can also be achieved via an adaptive feedback law that adjusts the desired pressure trajectory in time based on a target VT (V_{T}-target), V_{T} remaining to reach the target and/or excess VT (V_{T}_error), and possibly also the lung characteristics that are estimated for the particular patient. The lung characteristics could be resistance (R) and compliance (C), for example. In some embodiments, the adaptive feedback law may include information about the patient's breathing efforts (magnitude, duration, inspiratory time, etc.) and adjust the desired pressure trajectory in real-time to support spontaneous breaths and minimize patient-ventilator dyssynchrony. In such embodiments, the ventilator includes real-time, adaptive setting of a pressure trajectory, real-time parameter estimation, spontaneous breathing detection and adjustment, dyssynchrony detection and adjustment.

Figure “Coupling” shows an embodiment where the different controllers are coupled and where the desired commands either enter the system or are generated.

For example, FC and OC can be combined into one controller due to the coupling between the oxygen concentration and the flow. Indeed, all controllers can be combined into one MIMO (multi-input multi-output) controller, since it (ventilator air circuitry) is one system and pressure, flow, and oxygen concentration of the circulating air are inter-related.

In other embodiments, a controller may operate according to an optimal control law (and/or nonlinear and/or adaptive and/or robust). In other embodiments, multiple controllers may be grouped together. For example, multiple single-input single-output (SISO) controllers may be lumped together into a multi-input multi-output (MIMO) controller. For example, pressure and oxygen concentration of mixed air or pressure and flow can be simultaneously controlled. This MIMO controller could actuate valves on the respective source air supply lines. Other configurations of groupings of controllers are also possible to form different MIMO feedback systems.

In some embodiments, the ventilator 200 is a “smart” ventilator, which can be used by lay operators. As such, anyone can use at least the ventilator anywhere, at least for non-invasive breathing assistance, and may be able to user the ventilator invasively. Smart ventilators may include ambient sensors for detecting air quality (temperature, humidity, particulate matter (e.g., pollutants, pathogens (virus/bacteria/fungus)), or leak, and serve as input to one or more controllers to actuate blowers, motors, filters, UV lights, heaters, coolers, and humidification systems or devices in order to improve air quality being delivered to the patient. This is especially important for COPD and asthma patients, who are more vulnerable to exacerbation with air pollution.

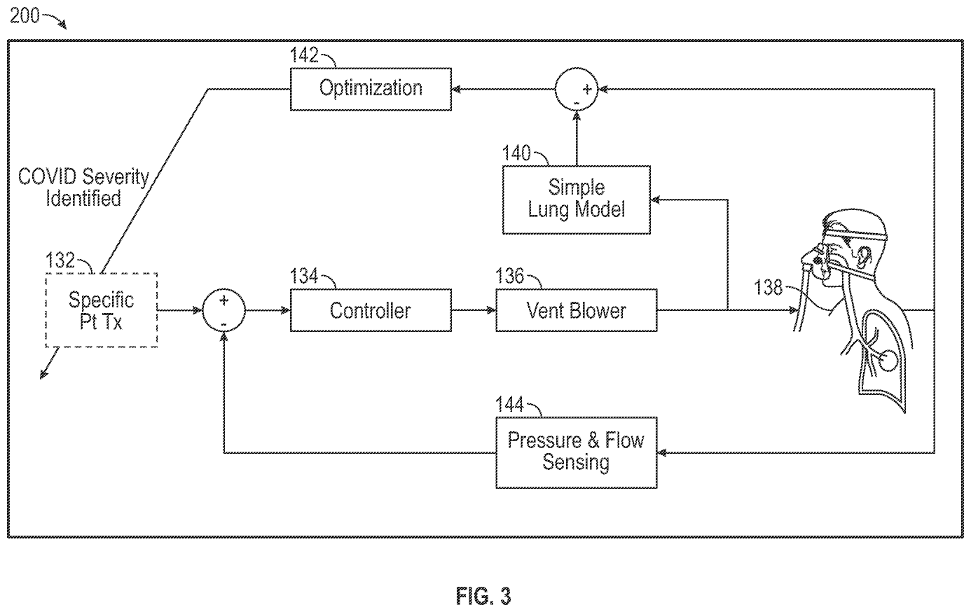

The ventilator 200 includes or can be updated/modified by including various components and software modules as described above. An example of a smart (s-series) ventilator is a ventilator configured for non-invasive estimation of the plant (lungs and tubing) physical characteristics as shown in FIG. 3. The goal here is to non-invasively estimate the level of severity level of a respiratory disease. For example, the severity of a coronavirus (e.g., COVID-19) or flu virus infection (“COVIDness”), the severity of acute respiratory syndrome (“ARDSness”), and/or the severity of chronic obstructive pulmonary disease (“COPDness”), among others, can be estimated and used to control ventilator variables. In some embodiments, the disease (e.g., COPD vs. ARDS. vs. COVID) is used to select or adjust target or desired setpoints or to select whether and how to control flow, pressure, or oxygen, or some combination thereof. In other embodiments, again, a MIMO controller can be used and the flow, pressure, and oxygen can each, or as groups, be controlled accordingly.

FIG. 3 depicts an embodiment of a smart version of the ventilator 200. The ventilator 200 includes a blower 136 controllable by a controller 134 to provide breathing support to a patient 138. The controller 134 is programmed to operate the blower 136 or valve (not shown) based on a target or desired setpoint determined by outputs of a mathematical lung model 140 and an estimation algorithm 142 used to assess mathematical lung model coefficients via a computer/board (not shown). The controller 134 receives outputs from the model 140 and sensors 144. The sensors 144 measure, e.g., ventilator variables (e.g., pressure, flow rate, oxygen concentration) and/or patient variables. Variable values are compared to one or more setpoints 132 to estimate errors (if any) and adjust the blower 136 or valve and/or other variables (e.g., oxygen concentrator settings).

For example, sensed measurements are compared to mathematical lung model outputs (e.g., pressure, volume, oxygen concentration or partial pressure, temperature, etc.) and the resulting error is used as an input to an optimization routine or algorithm 142. This optimization algorithm 142 enables the fine-tuning of the mathematical lung model 140 (specifically its parameters or equation coefficients) in real-time so the model response to a certain stimulus (e.g., airway pressure, flow, oxygen concentration, etc.) matches the lung response given the same stimulus. This task may be accomplished iteratively via the optimization algorithm 142, where the search space equals the number of parameters to be identified. The optimization algorithm 142, for example, minimizes an objective function. The objective function, in one embodiment, is the error between the model 140 and the patient's measured output. The output of the optimization (lung model parameter values or changes) can be used to assess the disease state of the patient, or severity level of respiratory disease (e.g., “COVIDness”) as aforementioned. This disease state can be used to select the patient-specific target or desired ventilator settings (or changes to them), resulting in a patient-specific or personalized therapy. In some embodiments, the estimation outputs may be put into an inference algorithm or classification algorithm before target or desired setpoints are selected. Though oxygen control and sensing are not shown in FIG. 3, they are envisioned in other embodiments along with temperature control and sensing and carbon dioxide sensing.

In some embodiments, the ventilator 200 and an associated controller are configured to receive measurement data from sensors that measure patient variables including physiological parameters of the patient. This patient measurement data may be used in conjunction with sensors that measure ventilator variables to control and/or optimize breathing support. Furthermore, the patient measurement data can be used to construct, adjust or update the model 140 or any other model.

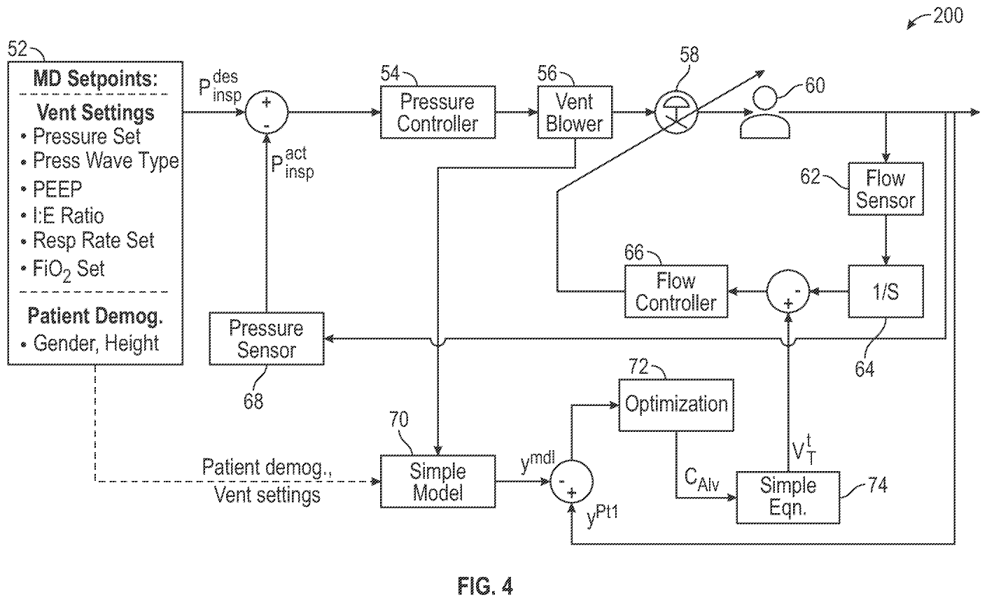

FIG. 4 shows an alternate embodiment of the smart ventilator 200, whereby a clinical or trained operator can select initial setpoints 52 for the ventilator and provide the patient demographics for initializing a simple lung mathematical model 70. This embodiment is not intended to be comprehensive or limit the presence of other sensors and controllers.

The ventilator 200 in this embodiment provides feedback control of both pressure and flow rate of air provided to a patient 60. This smart ventilator 200 includes a vent blower 56 (or pressurized air through other means) controlled by a pressure and/or flow controller(s) 54, and a valve 58 controlled by a flow rate controller 66. A flow sensor 62 measures flow rate. Set points 52 and pressure measurements are input to the model 70 of the patient (e.g., lung model), and an optimization routine that outputs a desired flow rate. The desired tidal volume is compared to the calculated tidal volume after integrating the measured flow rate from the flow sensor 62, and the flow controller 66 adjusts the flow rate, and hence volume, to the patient accordingly. In another embodiment, the comparison results feed into an adaptive control law to adjust the pressure trajectory for every patient at every time step.

The ventilator 200 in this embodiment is also configured to execute an optimization routine, which includes inputting patient demographics (and optionally also patient variable measurements) to the model 70. The model output is input to an optimization algorithm, which produces desired flow rate values. The desired flow rate values are compared to flow sensor measurements, and the flow controller 66 adjusts valve settings (e.g., percentage closed, angle of opening, etc.) to reduce or eliminate any error.

In FIG. 4 the optimization routine 72 can fine-tune a parameter that represents or is related to the compliance of the lungs (or the compliance of the alveoli CAN (air sacs) of the lungs). For example, the routine uses a simple equation 74, such as a constitutive equation, to obtain the desired tidal volume for the patient. An example of such a constitutive equation is volume equals the product of capacitance and pressure.

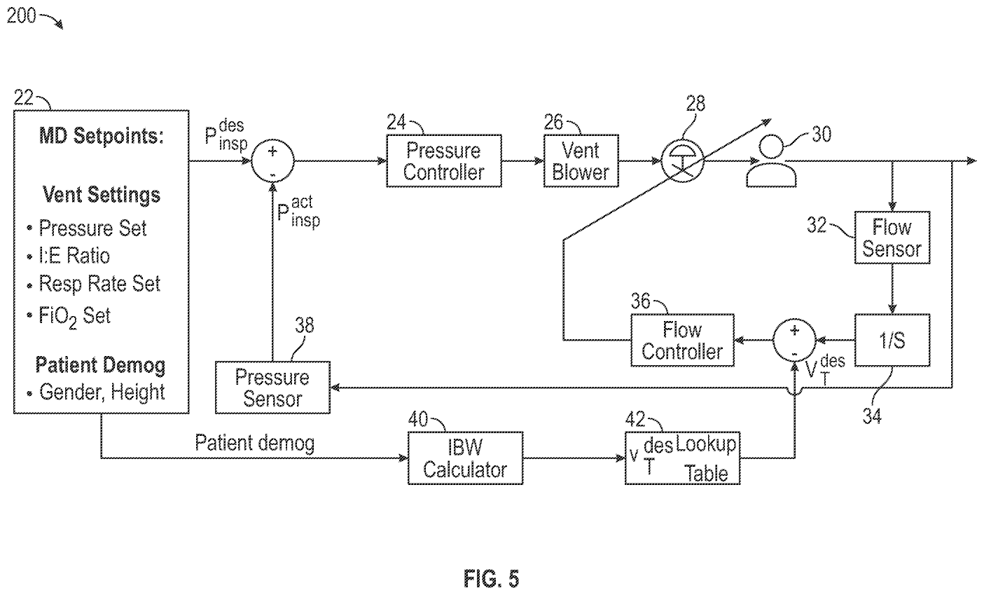

FIG. 5 shows another embodiment of the ventilator 200, which also provides feedback control of air supplied to a patient 30. In this embodiment, the ventilator 200 includes a vent blower 26 (or other sources or pressurized air) controlled by a pressure controller 24, and a valve 28 controlled by a flow rate controller 36. Pressure measurements (by a pressure sensor 38 that could be on the inspiratory or expiratory lines) of air supplied to the patient are provided as feedback to the pressure controller, which adjusts vent blower (or valve) settings based on a comparison between measured pressure and setpoints 22. A flow sensor 32 measures flow rate. Demographic information is provided to an ideal body weight (IBW) calculator 40, which uses a lookup table 42 or other means to determine lung volume and a desired tidal volume (integral of flow) based on the patient demographics. The desired tidal volume is compared to calculated tidal volume from the measured flow rate, and the flow controller 36 controls a valve 28 based on the comparison (e.g., an error value).

In FIG. 5, for example, the patient demographics (gender, height) entered as setpoints 22 are fed to the IBW calculator 40, which uses gender and height to calculate the ideal body weight of a person. The ideal body weight is then used as input to a tidal volume desired lookup table 42 mapping ideal body weight to (desired) tidal volume values based on clinically recommended tidal volume per kilogram of ideal body weight, such as 6 ml/kg. Extending this approach, one can use demographic or known/measurable patient variables as inputs to formulas, models, or calculators to obtain the desired volume, flow, pressure, O₂, CO₂, temperature, and/or other variables.

Also, if the FC and OC are combined into one, the demographic input and the desired ventilation protocol to be followed (ARDSnet low tidal volume ventilation, i.e., 6 ml/kgIBW, etc.) can dictate the target tidal volume. In addition, the information on the I:E ratio and RR (respiratory rate in beats per min) can further dictate a desired flow (per number of patient to be ventilated) that ensures that the tidal volume is not exceeded.

In some smart ventilator modes, the number of required entries are minimal to the user. For instance, default values of demographics, setpoints and initial values can be assigned. Lung characteristics can be estimated from measured variables (pressure and flow) and some of the default values can be adjusted accordingly.

In other smart ventilator modes, the ventilator's user interface would guide the lay operator through putting a mask or helmet on a patient. The ventilator would automatically set a safe pressure to start ventilation, estimate lung characteristics, and auto-adjust the pressure trajectory (while limiting volume). In some embodiments, the ventilator would estimate volume leak and auto-adjust. In some embodiments, the ventilator's user interface would guide the lay operator (optionally) through entering some basic demographic information (adult, pediatric, or infant patient), age, gender, or height prior to the ventilator's auto-setting a safe pressure to further individualize the safe pressure setpoint or tidal volume limit. Such smart ventilation modes are suitable for home, pandemic, war, natural disaster, and other such conditions, where anyone can ventilate a person in need. Such smart ventilators could be strategically placed to increase healthcare accessibility in urban or rural settings. They could be placed, for instance, in buildings (hotels, clinics, schools, government locations, etc.) or other transportation media (cars, trains, planes, buses, boats, vans, etc.) where other devices, such as defibrillators, are also stored. They could be placed in small medical hubs, sheds, or pods (e.g., MediPod) which house like-devices and are available for emergent or urgent use. Such hubs could include UV, O₃, and other disinfecting agents, disposables, as well as electrical charging stations. These smart ventilators could also be flown to places in need via drone (e.g., AirAssist).

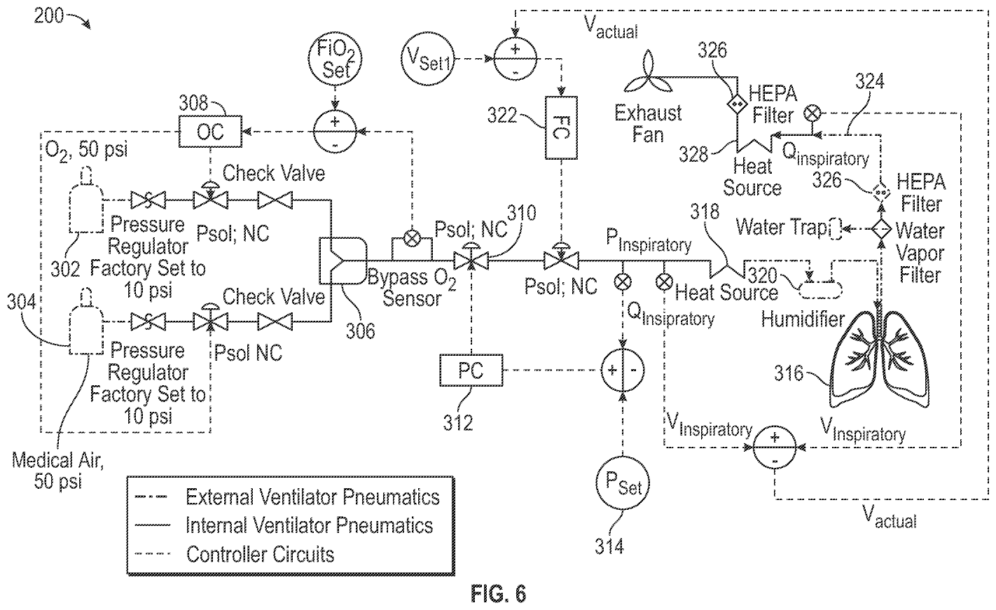

A hydraulic map of an embodiment of the ventilator 200 is shown in the ventilator hydraulics diagram of FIG. 6. Oxygen and medical air lines are fed through flow lines from an oxygen source 302 and a medical air supply 304 by oxygen and air at, e.g., 50 psi. Pressure regulators with venting are then introduced to reduce the pressure down to an acceptable level (around 1-2 psi) for mixing and for controlling the pressure and delivered oxygen concentration down further in an appropriate manner (as per physician's settings) in order to be delivered to the patient.

The reduced pressure gases are then mixed in a tube or chamber 306 where the oxygen concentration is then measured. The concentration can also be calculated based on the flow rates of the gases and the known air concentrations in both the air and oxygen lines (or tanks). The air's oxygen concentration can be calculated from desired medical air and oxygen flows that were pre-determined from target tidal volume and number of patients to be ventilated on the same ventilator. An oxygen controller 308 takes the difference between this measured, or calculated, oxygen concentration and a set inspired fraction of oxygen, and based on the difference the oxygen controller 308 adjusts the proportion of oxygen accordingly. The same controller 308 may also control a medical air PSol 310 where the actuation is the opening of an orifice allowing the gas to flow. Controlling the pressure in the medical air line can also be accomplished via an independent pressure controller 312 using measured pressure P mix as a feedback sensory element. This measured pressure may be provided by a pressure sensor 314.

The air reaching the patient (represented by lungs 316) is heated by a heat source 318 and humidified by a humidifier 320. The heated and humidified air should be at the concentration and pressure that are for normal ventilator operation, or ones set by a physician or operator. For this purpose, the pressure controller (PC) 312 may be designed and implemented to actuate the PSol 310 (which can be a proportional type valve) so the gas desired pressure time trajectory reaching the patient will be as dictated by the physician. A flow controller(s) 322 ensures that the right tidal volume reaches the patient. Since two closed-loop controllers are working concurrently, and in the case of multi-patient ventilation, then if the demands are high, then it is possible that the resulting gas in the patient(s)'s lungs may be not be exactly what the desired pressure and tidal volume are, however, they will be within acceptable ranges. This is offset by the important fact that both pressure and volume are controlled in the patient, and as such diminishing the need for physician/therapist's frequent intervention

On an expiratory line 324, provisions may be in place to filter and kill exhaled aerosol bacteria/viruses (e.g., via HEPA filters 326 and a heater 328), and a flow sensor is used in order to assess expired air, so when subtracted from inspired volume it gives an indication of breath stacking leading to intrinsic PEEP, which is an undesirable condition if left unchecked. The ventilator hence can provide an alarm, advise on I:E ratio or other settings that are entered by the physician, or adjust the target or desired I:E ratio or other settings. In some embodiments, there may be an on/off solenoid or 3/2 pneumatic valve on the inspiratory and/or expiratory lines or connected to y-shaped tubes in the inspiratory and/or expiratory lines which can route ambient air to/from the patient in emergency situations including but not limited to power and battery failure.

In non-invasive versions, the ventilator 200 may include a face mask or a helmet. The mask is small, but it dries the throat, irritates the skin, and is prone to leaking air. A helmet would eliminate the dryness and irritation; however, its size is bigger than that of a mask as the helmet envelopes the whole head.

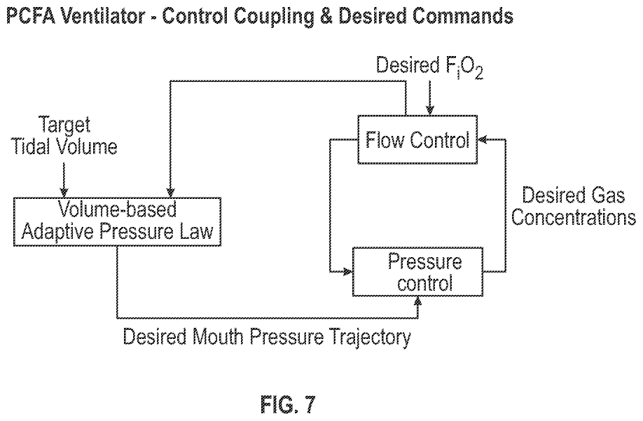

In an embodiment, pressure, flow, and O₂ concentration can be structured as in FIG. 7. A desired tidal volume target is provided to a volume-based adaptive pressure trajectory adjustment law, which outputs the desired pressure at the patient mouth. This desired pressure at the patient mouth is provided to a pressure controller, which receives input of mixed air flow and in turn commands desired gas flows and controls pressure at the mouth. The desired gas flows based on pressure control effort (which could be a pressure control error or a transfer function or other relationship between pressure and flow), together with a desired fraction of inspired oxygen (FiO₂), are provided to the oxygen and medical air flow controllers. These flows are then used to calculate the volume of air delivered to the patient, which is also used in the volume-based adaptive pressure trajectory law.

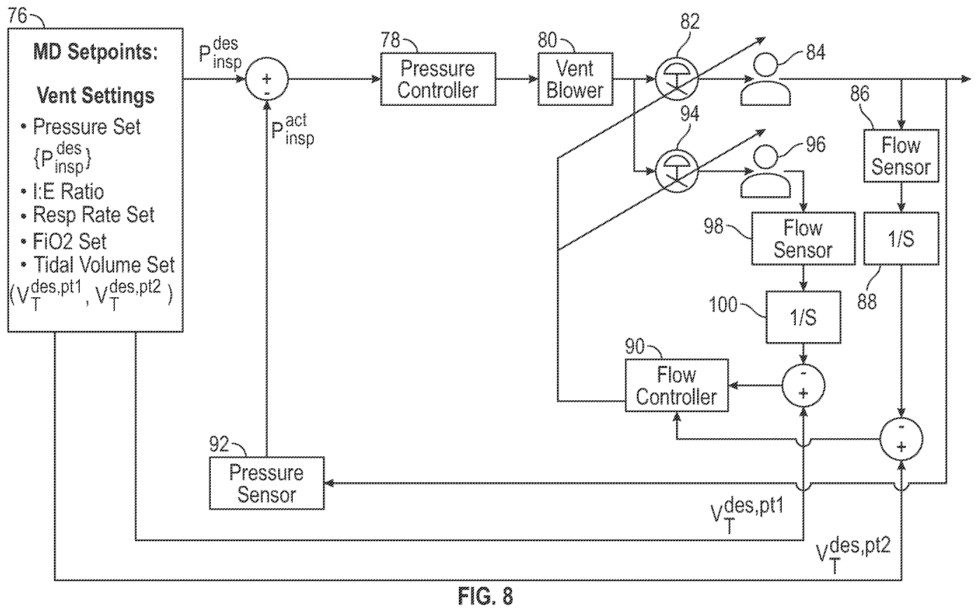

In other embodiments, the ventilator 200 may include additional components to support multi-patient ventilation as in FIG. 8. Examples of additional components to support multi-patient ventilation include inspiratory and expiratory line tubing, a Psol, and a flow sensor. For example, the ventilator 200 includes a vent blower 80 (or other sources or pressurized air) controlled by a pressure controller 78 (which can control a valve or valves, or a blower), and valves 82 and 94 controlled by a flow rate controller 90. Pressure measurements (by a pressure sensor 92 that could be on the inspiratory or expiratory lines) of air supplied to patients 84 and 96 are provided as feedback to the flow controller 90, which adjusts vent blower (or valve) settings based on a comparison between measured pressure and setpoints 76. A flow sensor 86 is connected to the patient 84, which sends a sensed flow signal to an integrator 88 to obtain the volume, and adjusts (as needed) the valve 82 (e.g., a butterfly valve or PSol) to control flow to the patient 84. Likewise, a flow sensor 98 is connected to the patient 96, which sends a sensed flow signal to an integrator 100 to obtain the volume, and adjusts (as needed) the valve 94 to control flow to the patient 96.

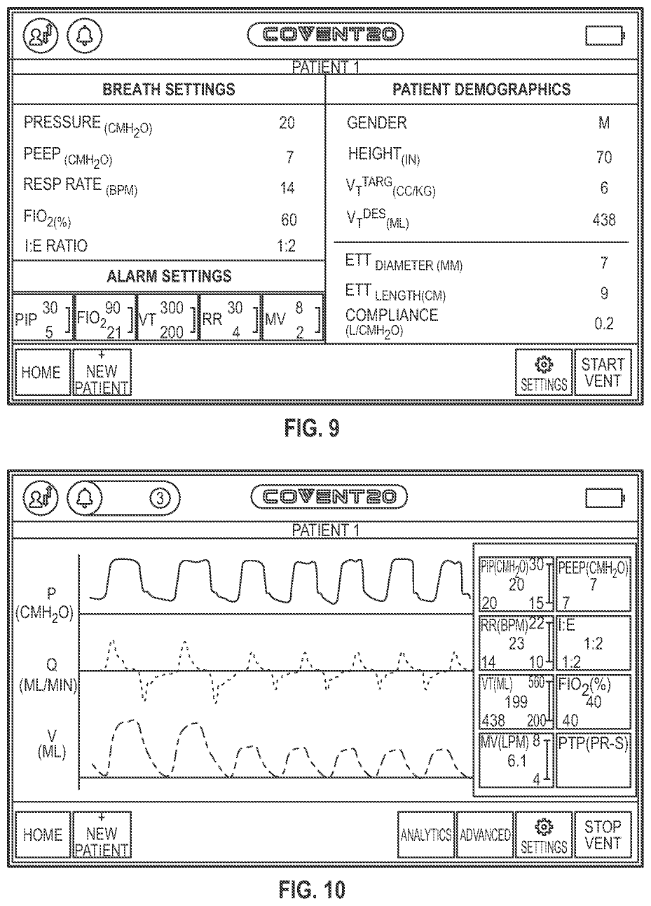

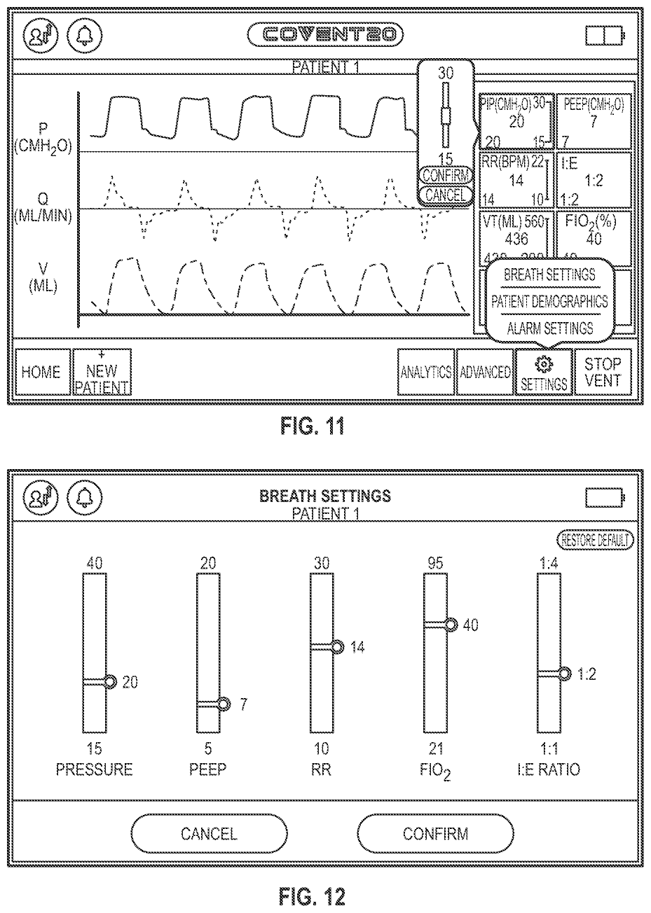

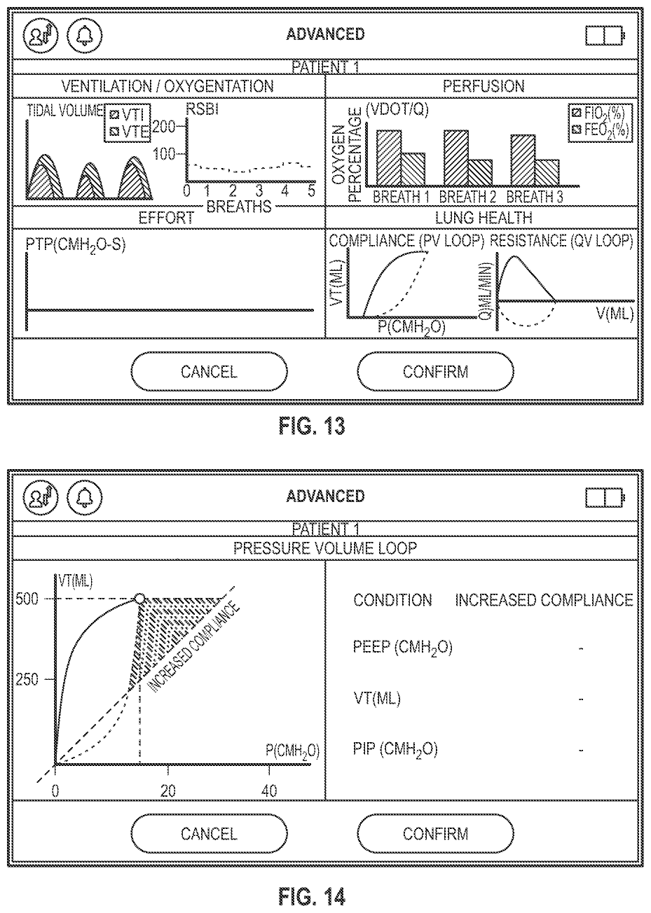

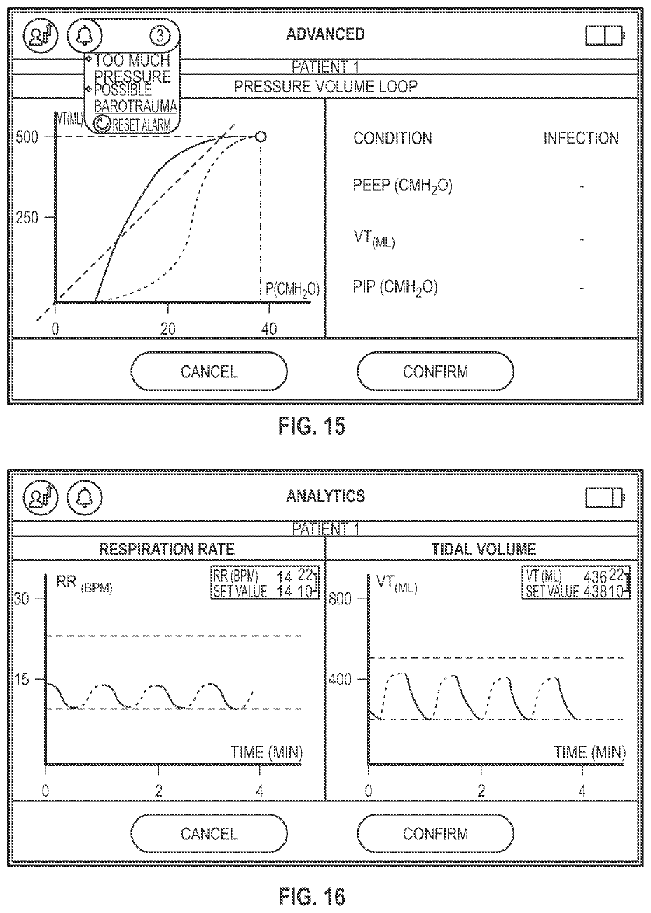

FIG. 9, FIG. 10, FIG. 11, FIG. 12, FIG. 13, FIG. 14, FIG. 15 and FIG. 16 show examples of displays that can be generated by the ventilator 200 or in conjunction with ventilator. These “screen shots” provide information to a user (e.g., in real time), such as current settings and patient variables. This information may be in the form of waveforms or numerics, or in any other suitable form. The displays may also provide information and present options and a user interface for changing/adjusting settings and/or alarm limits. These displays may additionally show measurements together with their respective alarm limits and related vent or breath settings. These displays may allow for editing values in place or navigating to new displays specifically for changing/adjusting settings. These displays may additionally include advanced features, such as outputs of real-time parameter estimation of lung health (e.g., resistance, compliance, diaphragmatic force, muscle pressure, etc.), real-time assessment of inspiratory or expiratory tidal volume, and/or real-time assessment of fraction of oxygen in inspired or expired air. These displays may additionally include plots that help characterize or infer information about lung health and/or patient breathing effort, such as pressure-volume (P-V) loops, flow-volume (Q-V) loops, pressure-flow (P-Q) loops, pressure-time product (PTP), pressure momentum, etc. These advanced displays may organize the user interface into different categories for better targeted or goal-driven therapeutic management, such as ventilation, oxygenation, perfusion, lung health, and patient effort/activity or dependence/reliance on the ventilator. Further, these displays may include analytics, trends, or temporal information of measurements and assessments over time, over breaths, or over portions (phases) of breaths. For instance, inspiratory and expiratory tidal volume can be used to identify breath stacking or intrinsic PEEP. For instance, high expired oxygen concentrations or large changes in expired oxygen concentrations can be used to identify perfusion or gas exchange problems. Such trends may be displayed to the user to allow for detection of trends that occur over time or such trends could be identified by algorithms or artificial intelligence, the information about or detection of which could be presented to the user. Some of these displays may include reference lines or plots for a population of patients or for the individual, for the individual now or at a previous time, or for the individual over a previous time or breath period. Some of these displays may include design accents, tracings, highlighting, boldness, lines, or other indicators to draw attention to areas of concern on plots or trends that are harmful. For instance, a display can show lines or a shaded region as the distance of a PV loop from a diagonal line through the origin as increased or decreased compliance. Another example is a display that highlights in red flattened regions, pointed parts, or abnormalities of a PV loop as portions of overdistension of the lung, potential high pressure, or potential barotrauma.

In one embodiment, a software application is provided and may include various modules or components for performing the various methods, routines and algorithms described herein, as well as simulation and training functions. The software application may be a program or mobile app. The software application may include one or more ventilator software components from FIGS. 3, 4 and/or 5 in order to provide scenarios, simulations, or training, where the software would ventilate a simulated computer patient (which could be based on user input of patient attributes, such as gender, height, airway resistance, and/or lung compliance). The software application may display information from one or more displays from FIGS. 9, 10, 11, 12, 13, 14, 15, and/or 16.

The software application may be embodied in hardware within a ventilator, or may otherwise be in communication with the ventilator in order to, for example, collect patient data, ventilator operational data, setpoints, and/or any other relevant information. For example, the software application may be in communication with the ventilator via a direct connection (e.g., wired or wireless) or an indirect connection (e.g., via a network).

The software application may be configured to monitor a patient and/or ventilator operation and present information to a patient, operator or other user. In an embodiment, the software application may provide outputs in the form of recommendations, alarms, notifications, etc. regarding adjustments (to operation of breathing assistance) to the user, and/or issue commands to change settings to the ventilator.

For example, the software application can generate a notification and physician order in an electronic health record (for approval by physician) and generate a notification for another clinician (e.g., respiratory therapist) when the order is approved.

A similar application, or web or mobile application, includes one or more ventilator software components from FIGS. 3, 4 and/or 5 in order to recommend suggested ventilator settings for one or more patients. A similar application, or web or mobile application, includes one or more ventilator software components from FIGS. 3, 4 and/or 5 in order to provide a noninvasive assessment of lung condition, such as lung health, lung parameters, disease, active/spontaneous breathing, breathing effort, respiratory muscle strength, and others. Lung health includes, for example, COVIDness, ARDSness, COPDness, lung fibrosis, obstructive sleep apnea, or the like. In other embodiments, the software application can allow user-entered demographics and other variables of the patient and/or model-estimated parameters or model-output variables of a simulated patient (emulating a real patient) and invoke an AI program, like a trained neural network, to recommend the best ventilator settings for such a patient, based on trained patient data who would be categorized as the same class as the current patient. Such web or mobile applications may display information from one or more displays from FIGS. 9, 10, 11, 12, 13, 14, 15, and/or 16.

As noted above, the ventilator 200 may include various modular components or add-ons to customize the ventilator for different purposes and users. One such component is a detachable add-on device to make a ventilator suitable for ventilating multiple patients. The device includes an apparatus or housing, tubes, valves, a computer running a controller, sensors, and an input for number of patients (and optionally patient demographics so a desired tidal volume can be computed). The device has a tube fitting to the inspiratory air tube and the expiratory air tube of an existing ventilator. Air travels from the inspiratory air tube of the original ventilator through an enclosed internal tube and valve structure of the apparatus and leaves from one of several inspiratory air tube lines. The air returns from the patient(s) though an expiratory tube line and through a network of tubing that merges to the single expiratory air tube of the original ventilator. This device would allow fewer ventilators to be used to safely support/ventilate more patients in case of a pandemic, when the number of sick patients overwhelms the ventilator supply in a given setting. The ventilator or this device can also come with a tape measure, so to allow the user to quickly measure the height of a patient and enter it as part of the computation of the desired tidal volume of the patient. In another embodiment of this device, the computer, controller, algorithms, and sensors need not be present. Instead, an apparatus housing tubes and manually-operated valves can be used.

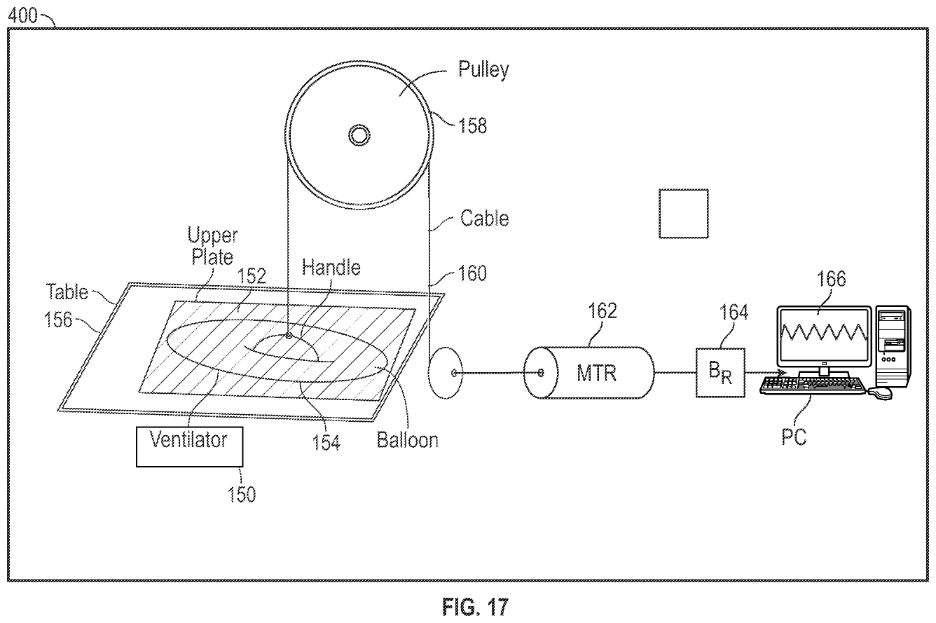

FIG. 17 depicts an embodiment of a device or system 400 (also referred to as a test rig) for testing the efficacy of ventilation by a ventilator, such as the ventilator 200. The test rig 400 is connected to a balloon or bellows 154, or other simulating means that can be used to exhibit similar inflation behavior as human lungs. Though not shown in the drawing, the airway resistance of the lungs (upper airway, dead space, etc.) can be represented with a resistor (restrictor), possibly at the mouth of the balloon/bellows 154, while the compliance (capacitance) characteristics of the lungs can be represented with one or more springs with known stiffness, possibly affixed to an upper plate 152 and located between the upper plate 152 and lower plate or table 156. The balloon or bellows has an opening for access to a ventilator 150.

In order to emulate natural breathing (i.e., breathing without a ventilator, like emulating a patient waking up from sedation) the pleural space is represented with a computer-controlled motor 162 that provides negative pressure impulses or trajectories to the balloon/bellows. In one embodiment, as shown in FIG. 10, a pulley system 158 and cable 160 can be used to pull the balloon/bellows upper plate 152 upward. In another, a motor pulls up (or down) on the upper plate as to emulate patient-induced inhalation (or exhalation). So, a motor 162 with appropriate gearing can control “breathing” by controlling the cable 160, or by directly actuating the upper plate of a test lung, say, to provide with the commanded negative pressures. This enables the bellows/balloon to better simulate a “lung” by enabling it to get simultaneous but separate sources of breathing (1) by ventilator/machine, sometimes called mandatory breathing, and (2) by patient respiratory drive, sometimes called spontaneous breathing, and muscles simulated by the pulley system 158 and cable 160.

The test rig 400 is controllable by a processing device such as a computer 166, which controls the test rig by controlling a driver 164. The computer 166 is not so limited and can control the test rig in any suitable manner, such as by controlling the upper plate 152 as noted above.

Spontaneous breathing trials (SBT) can be facilitated with understanding that can be derived using the test rig 400, as would patient-ventilator asynchrony studies. Further, the resistance and compliance of the test rig 400 can be made to match a certain (real) patient. Parameters or variables (e.g., airway R and lung C) of the real patient can be obtained via system identification techniques, similar to the one described in the optimization of FIG. 4. Though not shown, it is understood that sensors may be placed between the ventilator 150 and the mouth of the balloon/bellows 154 to measure pressure, flow, oxygen, temperature, carbon dioxide, or the like. It is also understood that some embodiments may include additional sensors inside the balloon/bellows 154. The pulley system 158 with cable 160, together with a force gauge, strain gauge, or load cell, can be used to measure the muscle force applied to the upper plate 152 of the balloon/bellows 154 which causes it rise and the balloon/bellows to expand, thus simulating natural breathing. Other embodiments may omit the pulley system and have a motor strategically placed at the base of the upper plate 152 with a gear box and levers to lift the upper plate 152 in straight or angular motion.

In some embodiments, different orifices or donut-shaped balloons can be used to emulate different airway resistances (from Rp 5 to 1000 cmH₂O*s/L), different sets of springs and/or spring-positions can be used to emulate different lung compliances (from 0.5 to 100 mL/cmH₂O), and a bellows of up to 2L can be used to emulate the lung volume (tidal volume and functional residual capacity). In some embodiments, a ventilator motor may be programmed to simulate different patient breathing efforts such as a time-varying muscle pressure (diaphragmatic force), a “waking-up” muscle pressure (diaphragmatic force), amplitude-varying muscle pressure (diaphragmatic force), and a naturally disturbed muscle pressure (e.g., cough, hiccup, etc.).

In some embodiments, R and C may be manually adjusted by the user, while in other embodiments R and C may be adjusted upon user selection on a graphical user input or via command. Optionally, or additionally, R and C groupings may be used to simulate different “diseases,” e.g., acute respiratory distress syndrome, emphysema, COPD, asthma, or coronavirus. These R and C groupings may be shown to the user for manual adjustment or the test rig may auto-adjust settings and emulate the disease condition.

In other embodiments, oxygen in the air delivered to the test rig (ventilator tester) may be filtered out and replaced with carbon dioxide to emulate gas exchange.

The following describes an embodiment of a flow sensor that may be used with or in the ventilator 200, or any other ventilator or other breathing device or system. The flow sensor is a bi-directional flow sensor that includes components of a conventional one-directional flow sensor. The flow sensor operates based on the pitot tube idea (Bernoulli approach), where the flow is obtained by using a differential pressure sensor and by employing the following equations:

$$Q = \sqrt{\frac{2 dP}{{ρ}_{gas}}} \cdot A ,$$

where Q is the volumetric flow rate of gas through a pipe in

$$\frac{{m}^{3}}{s} ,$$

ρ_{gas} is the density of gas in

$$\frac{kg}{{m}^{3}} ,$$

dP is the change in the pressure (of the flowing gas) between the two sensor ports in Pα, and A is the cross-sectional area of the pipe in m² and

$$A = \frac{π {d}^{2}}{4} .$$

This otherwise one-directional flow sensor is made bi-directional, by replacing a conventional stagnation pressure port with two pressure ports that point in the opposite direction from each other. The first port works based on the Bernoulli approach described above. In the event that flow reverses, the second port hosts the higher stagnation pressure since the centerline of the reversed flow goes into the mouth of the opposing port, hence naturally shutting off the first port.

Different configurations can be made so that the reverse flow stagnation pressure port is dominant. In one embodiment, two separate ports and tubes are included, each connected to a different pressure sensor. Both stagnation pressure ports are connected in a Y-shaped configuration, optionally with one-way valves so that the higher pressure always overwhelms the lower press stagnation pressure port so that the high pressure can reach the pressure sensor port. The static pressure port of the flow sensor remains the same and can thus be used in both flow directions.

Embodiments described herein address a number of problems that presently arise in conventional ventilation devices and systems. Current ventilators operate on positive pressure ventilation and operate under either Volume Control Ventilation (VCV) or Pressure Control Ventilation (PCV). In VCV, a desired volume waveform is dialed by the physician that is to be delivered to the patient. In VCV, pressure in the lungs was not monitored causing barotrauma. Barotrauma is a condition where excessive pressures in the lungs caused ischemia to the alveoli, hence debilitating gas exchange (that is needed to deliver O₂ to the rest of the body via blood).

In PCV, a desired pressure waveform, instead, is dialed by the physician. PCV addresses barotrauma issues due to tighter control of the pressures, but it could cause volutrauma, a condition where excessive air volumes were pushed into the lungs, which also compromised gas exchange. Current ventilators, while life supporting or sustaining, do not optimally solve the ventilation problem, especially because each patient's lungs are different from each other (due to disease, demographics, etc.).

Since providing air and O₂ to sick patients is needed, different combinations of PCV-VCV protocols emerged with practitioners who tried (and still do) to find the right balance of the ventilation types for each patient using such a machine. To address this problem, ventilator manufacturers devised different modes (12+) of VC and PV ventilations. Though they alleviated some of the inefficiencies leading to VILI, the correct sequence of ventilation types and modes for each patient's changing condition vexes critical care providers. The situation is exacerbated when the ventilated patient tries to breathe on his/her own, causing ventilator-patient dyssynchrony. Instead of forming universal standards for ventilating patients, different hospitals and physicians have formulated their own ventilation strategies. For example, in one hospital, a patient might be put on VCV while sedated, and then put on PCV when the patient starts to try to breathe on his/her own. At another hospital, the strategy for initiating and switching ventilator modes might differ.

Embodiments address the above problems by providing for, among others, capability for simultaneous and real time control of both pressure and volume (flow rate), as well as real time intelligent control of ventilator parameters as discussed above. The embodiments thus provide a solution to the above problems, while also providing for ventilators that can be easily customized for various purpose. In addition, the embodiments alleviate often burdensome (and in some cases insufficient) manual control of ventilator settings that is often required of physicians.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments of the invention, the practical application or technical improvement over technologies found in the marketplace, or to enable others of ordinary skill in the art to understand the embodiments described herein.

1. A breathing assistance system comprising:
a control unit configured to be connected to a device, the device configured to provide breathing assistance to a patient, the control unit configured to simultaneously control parameters that include:
a pressure of air supplied to the patient; and
at least one of a volume and a flow rate of the air.

2. The system of claim 1, wherein the control unit is configured to control the parameters to maintain a tidal volume of the air at or below a selected threshold.

3. The system of claim 1, wherein the control unit is configured to control the parameters according to one or more set points, the one or more set points including one or more values of at least one of the parameters, the one or more set points selected based on one or more individual properties of the patient, the one or more individual properties of the patient including at least one of:
a demographic;
a physical characteristic;
a type of a disease; and
a severity of the disease.

4. The system of claim 3, wherein the control unit is configured to automatically adjust the one or more set points based on a change in at least one of the one or more individual properties of the patient.

5. The system of claim 3, wherein the one or more individual properties of the patient are selected based on at least one of: user input, measurements of the patient, and a lung model.

6. The system of claim 1, wherein the control unit is configured to automatically adjust a pressure trajectory over time based on an adaptive control law.

7. The system of claim 1, further comprising a processing device configured to provide instructions to a user for operating the device.

8. The system of claim 6, wherein the adaptive control law periodically or continuously specifies adjustments to the pressure trajectory based on a deviation of a calculated tidal volume from a desired tidal volume.

9. The system of claim 1, wherein the control unit is configured to automatically adjust at least one of the parameters based on an adaptive control law, the adaptive control law specifying adjustments based on at least one of: a volume target, a lung parameter, a volume error, a spontaneous breathing condition, a dyssynchrony condition, pressure momentum and potential energy.

10. The system of claim 1, wherein the control unit is configured to receive sensor data related to air quality, and control actuation of one or more modules of controllers based on the sensor data to improve the air quality.

11. The system of claim 1, wherein the control unit is configured to control at least one of: an oxygen concentration of the air, a temperature of the air, and a humidity of the air.

12. The system of claim 1, wherein the device is configured to provide breathing assistance to a plurality of patients, and the control unit is configured to control the parameters independently for each patient.

13. The system of claim 12, wherein the device includes or is connected to one or more valves configured to direct air flow to each patient of the plurality of patients, and the control unit is configured to control the one or more valves.

14. A breathing assistance method comprising:
receiving sensor data related to a breathing assistance device at a control unit, the device configured to provide breathing assistance to a patient; and
simultaneously controlling parameters that include:
a pressure of air supplied to the patient; and
at least one of a volume and a flow rate of the air.

15. The method of claim 14, wherein the parameters are controlled to maintain a tidal volume of the air at or below a selected threshold.

16. The method of claim 14, wherein the parameters are controlled according to one or more set points, the one or more set points including one or more values of at least one of the parameters, the one or more set points selected based on one or more individual properties of the patient, the one or more individual properties of the patient including at least one of:
a demographic;
a physical characteristic;
a type of a disease; and
a severity of the disease.

17. The method of claim 16, wherein the control unit is configured to automatically adjust the one or more set points based on a change in at least one of the one or more individual properties of the patient.

18. A computer program product comprising a storage medium storing instructions executable by a processor to perform a method of facilitating use of a breathing assistance device by a patient, the method comprising:
receiving patient data from measurements of a patient, and device data related to operation of the breathing assistance device;
displaying information to the patient, the information including patient characteristics, and device settings including values of the parameters; and
displaying one or more recommendations to a user based at least one of the patient characteristics.

19. The computer program product of claim 18, wherein the one or more recommendations specify at least one of a value of a device setting and an adjustment to a device setting.

20. The computer program product of claim 18, wherein the one or more recommendations are based on at least one of: an assessment of a lung condition and an assessment of an individual patient disease.

21. A testing system for testing a breathing assistance device, comprising:
a testing assembly configured to emulate patient breathing activity, the testing assembly configured to be connected to the breathing assistance device;
one or more sensors configured to measure one or more properties of air flow produced by the testing assembly; and
a control unit in communication with the testing assembly, the control unit configured to control operation of the testing assembly.

22. The testing system of claim 21, wherein the control unit is configured to control operation of the testing assembly based on patient data acquired from at least one of:
an individual patient, and a group of patients.

23. The testing system of claim 21, further comprising a processing device configured to acquire sensor data from at least one of the testing assembly, the breathing assistance device and a patient, and display information to a user.

24. The testing system of claim 21, wherein the testing assembly includes a lung simulation device configured to simulate human lung behavior.
